(19) United States
(12) Patent Application Publication
(10) Pub. No.: US 2006/0241500 A1
(43) Pub. Date: Oct. 26, 2006

(54) METHOD AND DEVICE FOR DIRECT ULTRAHIGH SPEED CONVERSION FROM TIME SIGNAL TO SPACE SIGNAL

(76) Inventors: Tsuyoshi Konishi, Osaka (JP); Yoshiki Ichioka, Kobe-shi (JP)

Correspondence Address: MCGLEW & TUTTLE, PC, P.O. BOX 9227, SCARBOROUGH STATION, SCARBOROUGH, NY, 10510-9227, US

(21) Appl. No.: 11/381,626

(22) Filed: May 4, 2006

Related U.S. Application Data
(63) Continuation-in-part of application No. 10/311,791, filed on Dec. 18, 2002.

(30) Foreign Application Priority Data
Jun. 19, 2000 (JP) 2000/182667

(51) Int. Cl. A61B 6/00

(52) U.S. Cl. 600/476

(57) ABSTRACT

A signal light pulse and a reference ultra-short light pulse each having an appropriate spatially lateral width are launched simultaneously into an ultra-high-speed optical memory element from both sides of an optical axis thereof at appropriate angles with respect to the axis. Temporal waveforms of the signal light pulse and reference ultra-short light pulse are projected onto a plane. An interference fringe produced by interference between spatial projection images of two moving light waves corresponding to cross-correlation waveforms of the signal light pulse and reference ultra-short light pulse is retained in the ultra-high-speed optical memory element. A spatial distribution of self-diffracted light of the reference ultra-short light pulse produced in accordance with the spatial distribution of the retained interference fringe corresponding to the cross-correlation waveforms is imaged using an image forming lens and thus, is converted into a spatial distribution corresponding to the temporal waveform of the input signal light pulse.

### CROSS-REFERENCE TO RELATED APPLICATIONS

This application is a continuation in part application of U.S. patent application Ser. No. 10/311,791, filed on Dec. 18, 2002,

### BACKGROUND OF THE INVENTION

1. Field of the Invention

This invention relates to a method and apparatus for directly converting a time signal into a spatial signal at an ultra-high speed, more particularly to a method and apparatus for directly converting an ultra-short optical pulse time signal into a spatial signal at an ultra-high speed without any Fourier transform process or the like.

2. Description of the Related Art

In the field of optical communication, with the aim of real-time transmission of multimedia information including image data, sound data and text data, transmission capacity has been increased in recent years by time division multiplexing, wavelength division multiplexing or the like. However, since the signals used are in the form of time signals, as the transmission capacity becomes higher, it becomes needed to perform, at an ultra-high speed, conversion (encoding) of spatial information to be transmitted, such as an image, into time signals and development (decoding) of information in the form of time signals into spatial information.

An indirect method for converting between a time signal and a spatial information signal at an ultra-high speed based on the spectroscopic technology was proposed in a Reference 1 (Opt. Spectrosc., Vol. 57, pp. 1-6) in 1984. The method is advantageous in that conversion can be achieved without any dynamic device, but is disadvantageous in that any signal after conversion is only in the form of Fourier-transformed signal. Therefore, in any processing after conversion, the processing needs to be performed via the Fourier transform, and the time signal cannot be directly processed.

Besides, a method for developing a time signal into a spatial signal using interference was proposed in a Reference 2 (Opt. Lett., Vol. 18, pp. 2129-2131) in 1993. The method is advantageous in that the time signals can directly developed into the spatial form of interference fringes, but is disadvantageous in that the resulting signals can only be in the form of interference fringes and thus are difficult to process after the development.

In the past, various types of methods for converting between a time signal and a spatial information signal at an ultra-high speed have been proposed. According to these conventional methods, however, direct conversion between the time signals themselves and the spatial signals is impossible, while conversion between a frequency distribution of the time signals and the spatial signals is possible.

### SUMMARY OF THE INVENTION

It is an object of the present invention to provide a method for directly converting a time signal into a complete spatial signal at an ultra-high speed without any Fourier transform process.

It is another object of the present invention to provide an apparatus for directly converting a time signal into a complete spatial signal at an ultra-high speed without any Fourier transform process.

A method of the present invention is for directly converting a time signal into a spatial signal at an ultra-high speed. The method comprises preparing a signal light pulse and a reference ultra-short light pulse, each having a first and second predetermined spatially lateral width, respectively; launching the signal light pulse and the reference ultra-short light pulse simultaneously into a surface of an ultra-high-speed optical memory element from both sides of an optical axis thereof at appropriate angles with respect to the axis; retaining an interference fringe produced by interference between moving spatial projection images of waveforms of time signals of the incident signal light pulse and reference ultra-short light pulse in the ultra-high-speed optical memory element; and converting a spatial distribution of self-diffracted light of the reference ultra-short light pulse produced in accordance with the retained interference fringe into a spatial signal output corresponding to the time signal of the original signal light pulse.

An apparatus of the present invention is for directly converting a time signal into a spatial signal at an ultra-high speed. The apparatus comprises an ultra-high-speed optical memory element that is capable of modifying a transmission characteristic or a refractive index thereof in accordance with light incident thereon and retaining the modified state; a signal light pulse launching unit launching a signal light pulse into a surface of the ultra-high-speed optical memory element at a predetermined angle with respect to an optical axis of the element, the signal light pulse having a first predetermined spatially lateral width; and a reference ultra-short light pulse launching unit launching, simultaneously with the signal light pulse, a reference ultra-short light pulse into the surface of the ultra-high-speed optical memory element from a side of the optical axis opposite to the signal light pulse at a predetermined angle with respect to the optical axis, the reference ultra-short light pulse having a second predetermined spatially lateral width, wherein an interference fringe produced by interference, in the surface of the ultra-high-speed optical memory element, between waveforms of time signals of the signal light pulse launched by the signal light pulse launching means and reference ultra-short light pulse launched by the reference ultra-short light pulse launching means is retained in the ultra-high-speed optical memory element, and a spatial distribution of self-diffracted light of the reference ultra-short light pulse produced in accordance with the retained interference fringe is converted into a spatial signal output corresponding to the time signal of the original signal light pulse.

### BRIEF DESCRIPTION OF THE DRAWINGS

FIG. 1 shows an arrangement of a time-to-spatial signal conversion optical system according to one embodiment of the invention.

FIG. 2 illustrates a process of time-to-spatial signal conversion in the time-to-spatial signal conversion optical system according to one embodiment of the invention.

FIG. 3 illustrates a process of generation of interference fringes due to interference between a signal light pulse and a reference ultra-short light pulse.

### DETAILED DESCRIPTION OF THE PREFERRED EMBODIMENTS

Now, a preferred embodiment of the invention will be described with reference to the accompanying drawings.

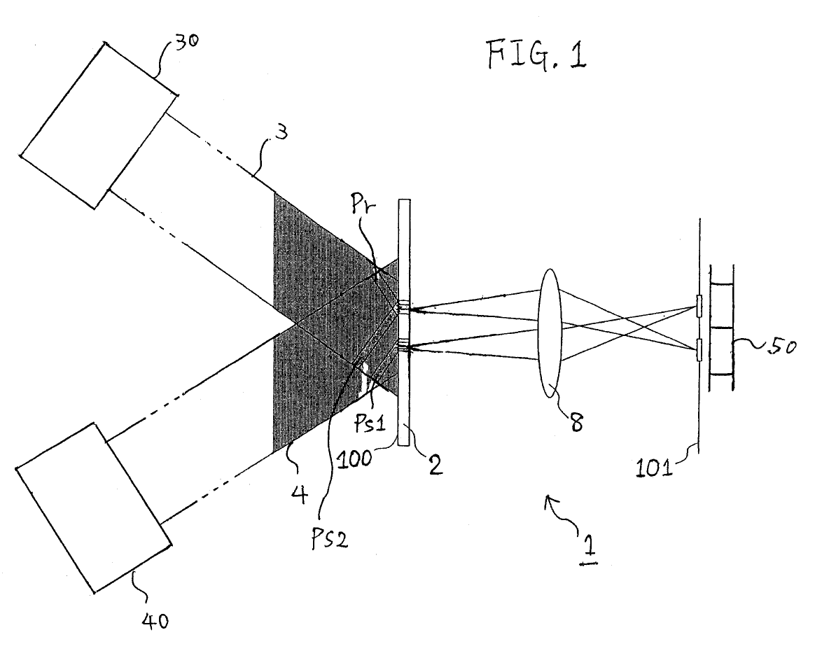

FIG. 1 shows an arrangement of a time-to-spatial signal conversion optical system according to one embodiment of the invention. In FIG. 1, a time-to-spatial signal conversion optical system 1 has a signal light pulse launching unit 30 which launches (or inputs) a signal light pulses 3 (indicated by Ps1 and Ps2 in FIG. 1), and a reference ultra-short light pulse launching unit 40 which launches a reference ultra-short light pulses 4 (indicated by Pr in FIG. 1), at an input side thereof.

The signal light pulse launching unit 30 launches a signal light pulse 3 into a surface of an ultra-high-speed optical memory element 2 at a predetermined angle with respect to an optical axis of the element 2. The signal light pulses 3 is a string of data pulses. For example, frequency and pulse width of the signal light pulses 3 is 40 GHz and 2 ps, respectively. This makes it possible to realizes an optical communication which has a very high bit rate such as 40 Gbps (bit per second) or more. The signal light pulse launching unit 30 comprises, as well known, a mode lock Laser and ultra-high speed optical modulator, for example.

The reference ultra-short light pulse launching unit 40 launches, simultaneously with the signal light pulse 3, the reference ultra-short light pulse 4 into the surface of the ultra-high-speed optical memory element 2 from a side of the optical axis opposite to the signal light pulse at a predetermined angle with respect to the optical axis. The reference ultra-short light pulses 4 are the lights to be reference of time, or reference wave. Frequency and pulse width of the reference ultra-short light pulses 4 is defined so as to make the pulse width shorter than a pulse interval of the signal light pulses 3. The reference ultra-short light pulses 4 are formed as well known based on single pulse which is separated from the signal light pulses 3.

The ultra-high-speed optical memory element 2 is capable of modifying a transmission characteristic or a refractive index thereof in accordance with light incident thereon, and retains (or memorizes) the modified state. As a result of this, the signal light pulses 3 is launched by the signal light pulse launching unit 30, and the reference ultra-short light pulses 4 is launched by the reference ultra-short light pulse launching unit 40, into the surface of the ultra-high-speed optical memory element 2. Then, each of waveforms of time signal (both of the pulses 3 and 4) are interfered at the surface of the ultra-high-speed optical memory element 2, thereby producing the interference fringe. The produced interference fringe is retained (or memorized) in the ultra-high-speed optical memory element 2. The retained interference fringe produces a spatial distribution of self-diffracted light of the reference ultra-short light pulse 4. The spatial distribution is a spatial signal output corresponding to the time signal of the original signal light pulse 3.

In FIG. 1, a light extracting unit 50 is provided on an output plane 101 of the time-to-spatial signal conversion optical system 1 with being associated with (or corresponding to) an output spatial distribution 9, 9. The light extracting unit 50 comprises an image pick-up device such as an array of photocells (or photo transistors) or a image shooting device such as a CCD, and electrically extracts the output spatial signals distributed in the output plane 101. Alternatively, as the light extracting unit 50, light-receiving ends of multiplexed optical waveguides or optical fibers may be provided on the output plane 101 with being associated with the output spatial distribution 9, 9′, thereby optically extracting the spatial signals. A plurality of the light extracting units 50, or a plurality of optical detector elements or a plurality of optical waveguides, are provided along the produced spatial distribution of the self-diffracted light of the reference ultra-short light pulse 4.

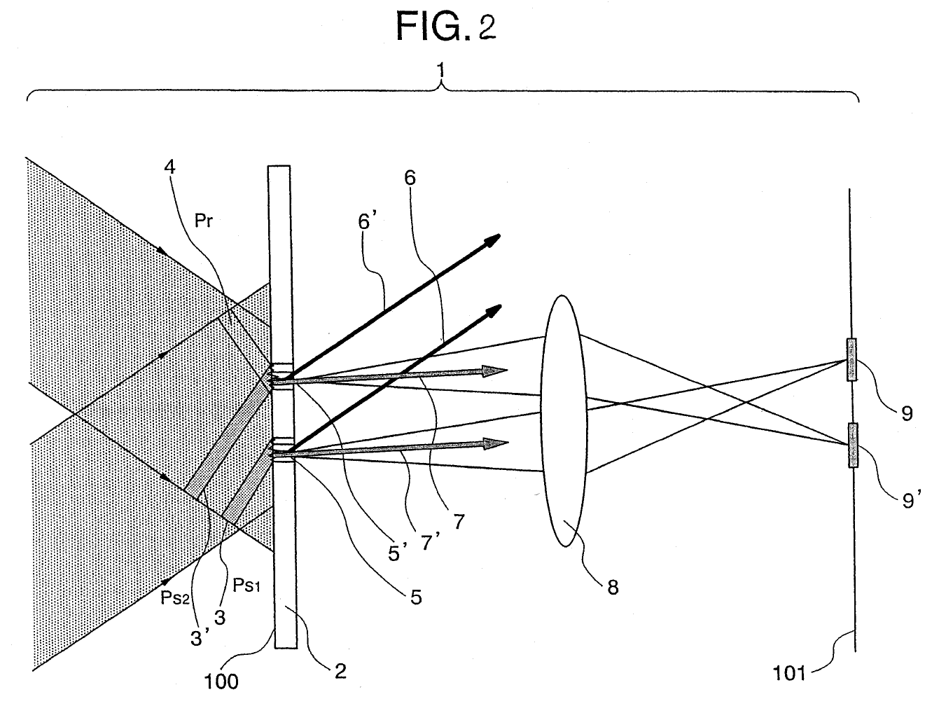

FIG. 2 shows an arrangement of a time-to-spatial signal conversion optical system, which realizes a method for directly converting a time signal into a spatial signal at an ultra-high speed according to the invention.

In FIG. 2, reference numeral 1 denotes a time-to-spatial signal conversion optical system, reference numeral 2 denotes an ultra-high-speed optical memory element, reference numerals 3 and 3′ denote signal light pulses (indicated by Ps1 and Ps2, respectively), reference numeral 4 denotes a reference ultra-short light pulse (indicated by Pr), reference numerals 5 and 5′ denote interference fringes, reference numerals 6 and 6′ denote transmitted light of the reference ultra-short light pulses 4, reference numerals 7 and 7′ denote primary diffracted light of the reference ultra-short light pulses 4 due to self-diffraction, reference numeral 8 denotes an image forming lens, reference numerals 9 and 9′ denote an output spatial distribution, reference numeral 100 denotes an incidence plane, and reference numeral 101 denotes an output plane.

The time-to-spatial signal conversion optical system 1 converts the received signal light pulses 3, 3′ in the form of time signals into spatial signals and outputs them on the output plane 101 in the form of the output spatial distribution 9, 9′.

For simplification, only two signal light pulses 3, 3′ to be converted are shown in this embodiment. However, any plurality of signal light pulses 3 may be launched in burst as far as the signal light pulses 3 can individually interfere with the reference ultra-short light pulse 4 to form their respective interference fringes on the ultra-high-speed optical memory element 2. Such signal light pulses 3 in burst include signals resulting from ultra-high-speed scanning of a binary image and signals resulting from multiplexing of multi-channel data.

The ultra-high-speed optical memory element 2 can modify optical characteristics thereof, such as transmittance (or absorptance) and refractive index, in accordance with the intensity of light incident thereon and retain the modified state. It may be a semiconductor device or a spatially modified liquid crystal optical element that has a multiple quantum well (MQW) structure.

In this embodiment, the ultra-high-speed optical memory element 2 comprises a semiconductor device having a multi-quantum well (MQW) structure, as described above. Due to the MQW structure, the ultra-high-speed optical memory element 2 uses carrier induced change in the exicitonic absorption to produce interference fringe by the signal light pulses 3 and the reference ultra-short light pulses 4. A produce speed of the interference fringe is 250 fs (femtosecond), for example. Such ultra-high-speed optical memory element 2 is known by “R. Takahashi, “Low-temperature-grown surface-reflection all-optical switch (LOTOS), “Optical and Quantum Electronics, vol. 33, 999-1017 (2001)”, which is incorporated herein by reference.

The signal light pulses 3, 3′ having an first appropriate spatially lateral width and the reference ultra-short light pulse 4 also having an second appropriate spatially lateral width are launched into the ultra-high-speed optical memory element 2 from both sides of an optical axis at appropriate angles with respect to the axis. Then, the incidence plane 100 is scanned at the speed of light with wave fronts of the signal light pulses 3, 3′ (Ps1, Ps2) and the wave front of the reference ultra-short light pulse 4 (Pr), which have reached the incidence plane 100 of the ultra-high-speed optical memory element 2, in opposite directions.

For example, in a case that length of the signal string (the signal light pulses 3, 3′) is 100 ps, a spatially lateral widths of the signal light pulses 3, 3′ should be 30 mm=100 ps×(3×10⁸ m/s). This makes it possible to convert the signal string collectively. This is similar on a spatially lateral widths of the reference ultra-short light pulses 4. An optical axis of the ultra-high-speed optical memory element 2 corresponds with an optical axis of a lens 8. Since there is needed reference of angle of the signal light pulses 3, 3′ and the reference ultra-short light pulses 4, in this embodiment, the optical axis of optical system. A size of the angle depends on a fineness of the interference fringe 5 which can be retained in the ultra-high-speed optical memory element 2. When a size of the angle becomes large, the fineness becomes small. In this embodiment, in a case that the angle is about 30 degree, the interference fringe 5 of about 1 mis produced. Due to incidence from both sides of the optical axis and scanning in the opposite direction, it becomes possible to make the reference ultra-short light pulses 4 and the signal light pulses 3, 3′ collide.

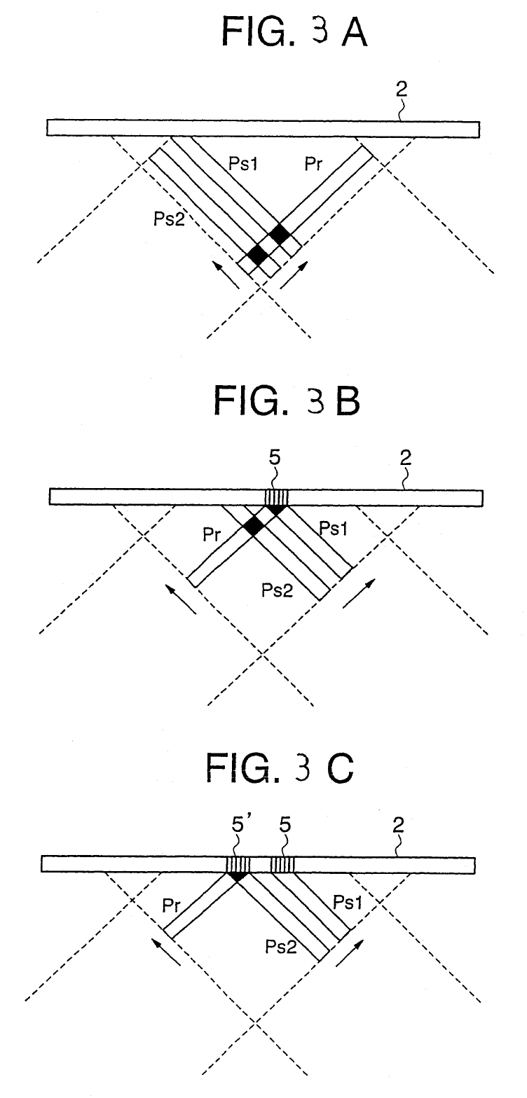

A pair of the light pulses Ps1 and Pr, which scans the incidence plane 100 in the opposite directions, interfere with each other at a spatial position where the wave fronts thereof simultaneously reach, thereby producing the interference fringe 5. And, a pair of the light pulses Ps2 and Pr, which scans the incidence plane 100 in the opposite directions, interfere with each other at a spatial position where the wave fronts thereof simultaneously reach, thereby producing the interference fringe 5′. A spatial distribution of the resulting interference fringes 5, 5′ corresponds to cross-correlation waveforms of spatial projection images of the interference light pulses. FIG. 3 shows a process of generation of the interference fringes 5, 5′.

The spatial position at which the reference ultra-short light pulses 4 collides with the signal light pulses 3, 3′ corresponds to the time position of the signal light pulses 3, 3′. Each of the pair is defined at each of positions of collision. Simultaneously reach means the state in which the reference ultra-short light pulses 4 collides with the signal light pulses 3, 3′. Since the collisions and interferences between the pulses 4 and 3, 3′ occur one after another, the interference fringe 5, 5′ is produced successively. Since collision part has an extent for the pulse width of the pulses 4 and 3, 3′, the interference fringe is spatially distributed. That is, the pulses 4 and 3, 3′ are projected onto the incidence plane 100, and then the spatial projection image is formed in accordance with the pulse widths. The projection image of the pulses 4 and 3, 3′ on the incidence plane 100 moves according to the motion of the pulses 4 and 3, 3′, or the spatial projection image moves. It corresponds for correlation (cross-correlation waveforms) to observe that the two waves advances in the opposite direction, interfere and separates.

FIG. 3A shows a state in which one end of the signal light pulse Ps1 has reached the surface of the ultra-high-speed optical memory element 2, and the signal light pulse Ps2 and the reference ultra-short light pulse Pr have not yet reached it. At this point in time, both positions of interference (two rectangular regions painted in black) between the pulses Ps1 and Pr and between the pulses Ps2 and Pr are distant from the surface of the ultra-high-speed optical memory element 2.

FIG. 3B shows a state in which one end of the reference ultra-short light pulse Pr reaches the surface of the ultra-high-speed optical memory element 2 and interferes with the signal light pulse Ps1 to produce the interference fringe 5, and the interference fringe 5 is retained (or the optical characteristics has been changed, as described later) in the ultra-high-speed optical memory element 2. At this point in time, although the pulse Ps2 has already reached the surface of the ultra-high-speed optical memory element 2, the position of interference (one rectangular region painted in black) between the pulses Ps2 and Pr is still distant from the surface of the ultra-high-speed optical memory element 2.

FIG. 3C shows a state in which the signal light pulse Ps2 and the reference ultra-short light pulse Pr cross each other in the surface of the ultra-high-speed optical memory element 2 and interfere with each other to produce the interference fringe 5′, and the interference fringe 5′ is retained in the ultra-high-speed optical memory element 2.

When the interference fringe 5 and 5′ is produced in the surface of the ultra-high-speed optical memory element 2, in the region where the interference fringe is produced, modification in optical characteristics including transmittance (absorptance) in accordance with the interference fringe pattern is attained and retained in an extremely short time (250 fs, for example). Therefore, at the point in time when the reference ultra-short light pulse Pr produces the interference fringe 5 and 5′, it is self-diffracted by the interference fringe to provide the transmitted light 6, 6′and the primary diffracted light (self-diffracted light) 7, 7′. The modification in optical characteristics is owing to the absorptance, state of spin, etc., for example, and depends on the ultra-high-speed optical memory element 2. The extremely short time means a range from sub-picosecond to several picosecond. The reference ultra-short light pulse Pr, which has a second predetermined spatially lateral width, forms the interference fringe 5 and 5′ at its front part, and the latter half is diffracted by the formed interference fringe Sand 5′. Accordingly, the reference ultra-short light pulse Pr is diffracted itself by the interference fringe 5 and 5′ which is formed by the reference ultra-short light pulse Pr itself.

Here, focusing only the primary diffracted light 7, 7′ on the output plane 101 by the image forming lens 8 can provide, on the output plane 101, the output spatial distribution 9, 9′ corresponding to time signal waveforms of the input signal light pulses 3, 3′. The lights which is separated in time (or which have phase difference) are outputted also separately in space. Accordingly, the output spatial distribution 9, 9′ is equal physically with development and distribution of time waveforms in space. The output spatial distribution 9, 9′ is a spatial signal, since information included in the signal is distributed in space.

The conversion performance of the time-to-spatial signal conversion optical system 1 according to the invention depends on the spatially lateral widths of the signal light pulses 3 and reference ultra-short light pulse 4, depends on the pulse width of the signal light pulses 3 and reference ultra-short light pulse 4, depends on the interval and the maximum number of the signal light pulses 3 in the burst. In particular, in response to a phase difference between the signal light pulses 3 and reference ultra-short light pulse 4, the position of the interference fringe produced in the surface of the ultra-high-speed optical memory element 2 is changed, and the output spatial distribution 9, 9′ on the output plane 101 is also changed in position. Thus, the condition of generating the reference ultra-short light pulse 4 is adapted to be changeable as various design values, so that the condition of producing the interference fringes in the surface of the ultra-high-speed optical memory element 2 can be appropriately controlled.

As described above, input signal light pulses of time signals can be converted into spatial signals. For example, if the input signal light pulses are time signals resulting from scanning of an image, the original image can be spatially developed on the output plane. Besides, if the input signal light pulses are time signals resulting from multiplexing of multi-channel data, the data for the individual channels can be output separately on the output plane.

The invention should not be limited to the embodiment described above, and many modifications and alterations thereto are possible. For example, only one lens is used for image formation in the above-described embodiment. However, if the output plane needs to be further distant from the ultra-high-speed optical memory element 2, a telecentric optical system including two lenses may be used.

As described above, with the method and apparatus for directly converting a time signal into a spatial signal at an ultra-high speed according to the invention, the time signal can be directly converted into the spatial signal at an ultra-high speed, rather than indirectly through a spectroscopic technology which is essential in conventional manners.

What is claimed is:

1. A method for directly converting a time signal into a spatial signal at an ultra-high speed, the method comprising:
preparing a signal light pulse and a reference ultra-short light pulse, each having a first and second predetermined spatially lateral width, respectively;
launching the signal light pulse and the reference ultra-short light pulse simultaneously into a surface of an ultra-high-speed optical memory element from both sides of an optical axis thereof at appropriate angles with respect to the axis;
retaining an interference fringe produced by interference between moving spatial projection images of waveforms of time signals of the incident signal light pulse and reference ultra-short light pulse in the ultra-high-speed optical memory element; and
converting a spatial distribution of self-diffracted light of the reference ultra-short light pulse produced in accordance with the retained interference fringe into a spatial signal output corresponding to the time signal of the original signal light pulse.

2. A method for directly converting a time signal into a spatial signal at an ultra-high speed according to claim 1, wherein the spatially lateral widths of the signal light pulse and reference ultra-short light pulse are large enough for the moving spatial projection images of the waveforms of the time signals of the signal light pulse and reference ultra-short light pulse incident on the surface of the ultra-high-speed optical memory element to interfere with each other in the surface of the ultra-high-speed optical memory element to produce the interference fringe.

3. A method for directly converting a time signal into a spatial signal at an ultra-high speed according to claim 1, wherein a condition of generating the reference ultra-short light pulse is adapted to be changeable to control a condition of producing the interference fringe.

4. An apparatus for directly converting a time signal into a spatial signal at an ultra-high speed, the apparatus comprising:
an ultra-high-speed optical memory element that is capable of modifying a transmission characteristic or a refractive index thereof in accordance with light incident thereon and retaining the modified state;
a signal light pulse launching unit launching a signal light pulse into a surface of the ultra-high-speed optical memory element at a predetermined angle with respect to an optical axis of the element, the signal light pulse having a first predetermined spatially lateral width; and
a reference ultra-short light pulse launching unit launching, simultaneously with the signal light pulse, a reference ultra-short light pulse into the surface of the ultra-high-speed optical memory element from a side of the optical axis opposite to the signal light pulse at a predetermined angle with respect to the optical axis, the reference ultra-short light pulse having a second predetermined spatially lateral width,
wherein an interference fringe produced by interference, in the surface of the ultra-high-speed optical memory element, between waveforms of time signals of the signal light pulse launched by the signal light pulse launching means and reference ultra-short light pulse launched by the reference ultra-short light pulse launching means is retained in the ultra-high-speed optical memory element, and a spatial distribution of self-diffracted light of the reference ultra-short light pulse produced in accordance with the retained interference fringe is converted into a spatial signal output corresponding to the time signal of the original signal light pulse.

5. An apparatus for directly converting a time signal into a spatial signal at an ultra-high speed according to claim 4, wherein the ultra-high-speed optical memory element comprises a semiconductor device having a multiple quantum well structure.

6. An apparatus for directly converting a time signal into a spatial signal at an ultra-high speed according to claim 4, further comprising:
a plurality of optical detector elements or a plurality of optical waveguides provided along the produced spatial distribution of the self-diffracted light of the reference ultra-short light pulse.
